(19) United States
(12) Patent Application Publication
(10) Pub. No.: US 2022/0370379 A1
(43) Pub. Date: Nov. 24, 2022

(54) CANNABINOID-CONTAINING ADDITIVE AND METHOD THEREFOR

(71) Applicant: PURE GREEN PHARMACEITICALS, INC., West Bloomfield, MI (US)

(72) Inventors: Stephen Goldner, West Bloomfield, MI (US); Ara Kirakosyan, Saline, MI (US)

(21) Appl. No.: 17/640,473

(22) PCT Filed: Sep. 4, 2020
(86) PCT No.: PCT/US2020/049326

§ 371 Date: Mar. 4, 2022

Related U.S. Application Data
(60) Provisional application No. 62/896,191, filed on Sep. 5, 2019.

(51) Int. Cl. A61K 31/05; A61K 47/44; A61K 47/24; A61K 47/36; A61K 31/12; A61K 31/4525; A61K 9/00; A23L 33/105; A23L 33/115; A23D 7/005; A23D 7/04; A23L 2/52

(52) U.S. Cl. CPC A61K 31/05; A61K 47/44; A61K 47/24; A61K 47/36; A61K 31/12; A61K 31/4525; A61K 9/0056; A23L 33/105; A23L 33/115; A23D 7/0053; A23D 7/04; A23L 2/52; A23V 2002/00

(57) ABSTRACT

A method of preparing a cannabinoid-containing additive includes preparing an oil mixture by combining a cannabinoid oil and a carrier oil, preparing a water base by combining water and an emulsifier blend, and combining the oil mixture and the water base to form a liquid cannabinoid emulsion. The emulsion may be dried to form a solid. The cannabinoidcontaining additive, either in the liquid or solid form, is combined with a food and beverage product.

### BACKGROUND

Cannabinoids are a class of active compounds derived from the Cannabis sativa, Cannabis indica, or Cannabis hybrid plant, commonly known as marijuana. A few known cannabinoids include cannabidiol (CBD), tetrahydrocannabinol (THC), such as delta-9-tetrahydrocannabinol (Δ9-THC) and delta-8-tetrahydrocannabinol (Δ8-THC), cannabigerol (CBG) , cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), tetrahydrocannabinolic acid (THCA), cannabinol (CBN), and cannabidiolic Acid (CBDA).

Cannabinoids, and in particular CBD, are used for medicinal purposes to treat disease or alleviate symptoms. Cannabinoids can be delivered into the body through inhalation, ingestion, or topical application. Delivery via ingestion is of particular interest due to detrimental effects associated with inhalation. Accordingly, delivery through food and beverages is desirable. One challenge, however, to incorporation of cannabinoids into ingestible food and beverage products is that cannabinoids are generally only very slightly soluble in water or are practically insoluble in water. Since many food and beverage products contain water or are water-based, there can be considerable difficulty to produce a stable cannabinoid-containing food or beverage product.

### SUMMARY

A method of preparing a cannabinoid-containing additive according to an example of the present disclosure includes preparing an oil mixture by combining a cannabinoid oil and a carrier oil, preparing a water base by combining water and an emulsifier blend, and combining the oil mixture and the water base to form a cannabinoid emulsion.

In a further embodiment of any of the foregoing embodiments, the cannabinoid oil is cannabidiol (CBD) oil.

In a further embodiment of any of the foregoing embodiments, the carrier oil is selected from the group consisting of coconut oil, olive oil, soybean oil, grapeseed oil, avocado oil, and combinations thereof.

In a further embodiment of any of the foregoing embodiments, the emulsifier blend is two or more emulsifiers selected from the group consisting of rhamnolipid, xanthan gum, lecithin, guar gum, quillaja saponin, karaya gum, and acacia gum.

In a further embodiment of any of the foregoing embodiments, the emulsifier blend is lecithin, karaya gum, and quillaja saponin.

In a further embodiment of any of the foregoing embodiments, the emulsifier blend is lecithin, acacia gum, and quillaja saponin.

In a further embodiment of any of the foregoing embodiments, the emulsifier blend is rhamnolipid, xanthan gum, and lecithin.

In a further embodiment of any of the foregoing embodiments, the emulsifier blend is rhamnolipid, guar gum, and lecithin.

In a further embodiment of any of the foregoing embodiments, by weight, at least half of the emulsifier blend is at least one of rhamnolipid or quillaj a saponin.

In a further embodiment of any of the foregoing embodiments, by weight, the emulsifier that is present in the emulsifier blend in is at least one of rhamnolipid or quillaja saponin.

A further embodiment of any of the foregoing embodiments includes sonicating the water base, and wherein the combining includes adding the oil mixture to the water base during the sonicating.

A further embodiment of any of the foregoing embodiments includes removing the water from the cannabinoid emulsion to form a dry powder.

A further embodiment of any of the foregoing embodiments includes either adding the cannabinoid emulsion to a food and beverage product or removing the water from the cannabinoid emulsion to form a dry powder and then adding the dry powder to the food and beverage product.

A further embodiment of any of the foregoing embodiments includes adding a bioenhancer, the bioenhancer selected from the group consisting of curcumin and piperine.

A cannabinoid-containing additive according to an example of the present disclosure includes, by weight percent 1% to 5% of an emulsifier blend, 1% to 15% of a cannabinoid oil, 8% to 12% of a carrier oil, and a remainder of water.

In a further embodiment of any of the foregoing embodiments, the cannabinoid oil is cannabidiol (CBD) oil.

In a further embodiment of any of the foregoing embodiments, the carrier oil is selected from the group consisting of coconut oil, olive oil, soybean oil, grapeseed oil, avocado oil, and combinations thereof.

In a further embodiment of any of the foregoing embodiments, the emulsifier blend is two or more emulsifiers selected from the group consisting of rhamnolipid, xanthan gum, lecithin, guar gum, quillaja saponin, karaya gum, and acacia gum.

In a further embodiment of any of the foregoing embodiments, the emulsifier blend is lecithin, karaya gum, and quillaja saponin.

In a further embodiment of any of the foregoing embodiments, the emulsifier blend is lecithin, acacia gum, and quillaja saponin.

In a further embodiment of any of the foregoing embodiments, the emulsifier blend is rhamnolipid, xanthan gum, and lecithin.

In a further embodiment of any of the foregoing embodiments, the emulsifier blend is rhamnolipid, guar gum, and lecithin.

A further embodiment of any of the foregoing embodiments includes a bioenhancer. The bioenhancer is selected from the group consisting of curcumin and piperine.

A consumer product according to an example of the present disclosure includes a food and beverage product including either a cannabinoid-containing additive comprising, by weight percent: 1% to 5% of an emulsifier blend, 1% to 15% of a cannabinoid oil, 8% to 12% of a carrier oil, and a remainder of water, or a dried powder of the cannabinoid-containing additive without the water.

### DETAILED DESCRIPTION

Cannabinoids are generally incompatible with food and beverage products. Disclosed herein is a cannabinoid-containing additive, as well as methods related thereto, that is relatively stable and compatible for incorporation into food and beverage products. Although tea and ice cream are used in examples herein, it is to be understood that this disclosure may be applicable to a wide variety of food and beverage products, including also alcoholic beverages, dietary supplements, and soft drinks.

The cannabinoid-containing additive is a liquid emulsion but can also be further processed to form a dry powder. Accordingly, the term “additive” used herein may refer to either the liquid or the solid (e.g., powder), the main difference between these being that the liquid contains water and the solid is substantially devoid of water. In the liquid form, the additive includes, by weight percent: 1% to 5% of an emulsifier blend, 1% to 15% of a cannabinoid oil, 8% to 12% of a carrier oil, and a remainder of water. If desired, the additive may contain other constituents, in which case the above constituents (aside from water) may be provided in the same ratio in parts by weight.

Cannabinoid oils are the purified extracts of cannabinoids from cannabis plants. For example, the cannabinoid oil is cannabidiol (CBD) oil, such as pure or substantially pure CBD. As will be appreciated, the cannabinoid oil may contain impurity levels of other cannabis plant constituents, such as terpenes or other types of cannabinoids. It is also envisioned, however, that the cannabinoid oil used herein contain a different cannabinoid than CBD and/or a mixture of cannabinoids selected from, but not limited to, cannabidiol (CBD), tetrahydrocannabinol (THC), such as delta-9-tetrahydrocannabinol (Δ9-THC) and delta-8-tetrahydrocannabinol (Δ8-THC), cannabigerol (CB G), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), tetrahydrocannabinolic acid (THCA), cannabinol (CBN), and cannabidiolic acid (CBDA).

The carrier oil is generally an edible oil, such as an edible medium chain triglyceride oil. For example, the carrier oil is selected from coconut oil, olive oil, soybean oil, grapeseed oil, avocado oil, and combinations thereof. Cannabinoids are fat-soluble and the carrier oil thus serves to strongly bind with the cannabinoid, thereby facilitating a stable emulsion.

The emulsifier blend is two or more emulsifiers selected from rhamnolipid, xanthan gum, lecithin, guar gum, quillaja saponin, karaya gum, and acacia gum. The emulsifier blend serves to stabilize the above oils in the emulsion and is dependent upon the hydrophilic-lipophilic balance (HLB) number of the emulsifiers in the blend. The HLB is an indicator of the ability of an emulsifier to stabilize an emulsion of a particular type. For instance, an HLB number that is very low (less than 3) is lipophilic that is soluble in oil but has poor stability for water. Inversely, an HLB number that is very high (greater than 18) is hydrophilic that is soluble in water but has poor stability for oil.

Rhamnolipid may be used to facilitate good antimicrobial activity, longer shelf life of the final product, control of agglomeration of fat globules and stability of product consistency, solubilization of flavor oils, and reduction in spattering in ice cream formulation.

The emulsifier blend herein contains a mixture of emulsifiers, which have different HLB numbers. For instance, the above emulsifiers have the following HLB numbers: rhamnolipid (>12), xanthan gum (>11), lecithin (about 8), guar gum (>12), quillaja saponin (>13), karaya gum (>11), and acacia gum (about 13.5). In one example, the emulsifier blend is lecithin, karaya gum, and quillaja saponin. In another example, the emulsifier blend is lecithin, acacia gum, and quillaja saponin. In an additional example, the emulsifier blend is rhamnolipid, xanthan gum, and lecithin. In another example, the emulsifier blend is rhamnolipid, guar gum, and lecithin.

In further examples, at least half of the emulsifier blend by weight is at least one of rhamnolipid or quillaja saponin, such as at least 55% or at least 80% (of the 1% to 5% in the additive).

In further examples, the additive additionally includes one or more bioenhancers. A bioenhancer is an agent that is capable of enhancing bioavailability and efficacy of the cannabinoid, without any pharmacological activity of its own at the dose used. As an example, the bioenhancer is selected from, but is not limited to, curcumin and piperine. The bioenhancer is generally provided in relatively small amounts. For instance, the additive includes from 1 milligram to 10 milligrams per 100 milliliters of additive, such as 5 milligrams.

Also disclosed is a method of making the additive. The method may be conducted at an ambient temperatures of approximately 23° C. in suitable vessels for handling the materials. The method includes preparing the oil mixture by combining the cannabinoid oil and the carrier oil. The combined oils may be subjected to mixing for a period of time that is sufficient to substantially homogenize the mixture. A water base is prepared by combining water and the emulsifier blend, and then the oil mixture is combined with the water base to form a cannabinoid emulsion.

In a further example, the water base is sonicated and the oil mixture is added to the water base during the sonication. If used, the xanthan gum or other gum may not completely dissolve, in which case the emulsion can be heated up to approximately 40° C. to facilitate dissolution.

The resulting cannabinoid emulsion is the liquid form of the cannabinoid-containing additive. If a solid (e.g., powder) form is desired, the emulsion is further processed in a drying step to remove the water. For example, the water is removed by freeze-drying or rotary evaporation. The removal of the water causes the crystallization of the constituents into solid form. The additive, either in its liquid form or its solid form, is then added to a food and beverage product to produce a consumer product. The food and beverage product thus includes either the cannabinoid-containing additive that comprises, by weight percent: 1% to 5% of an emulsifier blend, 1% to 15% of a cannabinoid oil, 8% to 12% of a carrier oil, and a remainder of water, or the dried powder of the cannabinoid-containing additive without the water. The amount of additive used selected in accordance with the desired dosing. For example, the dosing of the cannabinoid in the food and beverage product is from 0.1 mg to 100 mg of the emulsion or powder per gram of the food or beverage.

The following examples demonstrate additional non-limiting aspects of the disclosure. As will be appreciated, the examples herein can be scaled up or down in accordance with a desired output.

### EXAMPLE 1

### CBD Additive for Tea

An oil mixture is prepared with 5 milliliters of CBD oil and 10 milliliters of coconut oil. A water base is prepared by adding an emulsifier blend of 2 milligrams of quillaja saponin, 1 milligram of karaya gum, and 0.5 milligrams of lecithin to approximately 81.5 milliliters of distilled water. The water base is sonicated for 4 minutes with a 5-second on/off pulse, followed by a 3-minute break, followed by sonication for another 3 minutes with a 5-second on/off pulse. The resulting liquid emulsion has a pH of approximately 7.4. The liquid emulsion is dried by rotary evaporation to produce a dry powder. The powder is tolerant, or stable, in hot water that is used for tea.

### EXAMPLE 2

### CBD Additive for Ice Cream

An oil mixture is prepared with 5 milliliters of CBD oil and 10 milliliters of soybean oil. A water base is prepared by adding an emulsifier blend of 2 milligrams of rhamnolipid, 0.5 milligrams of lecithin, and 0.1 milligrams of xanthan gum to approximately 82.5 milliliters of distilled water. 5 milligrams of curcumin is also added to the water base. The water base is then sonicated for 4 minutes with a 5-second on/off pulse, followed by a 2-minute break, followed by sonication for another 3 minutes with a 5-second on/off pulse. The resulting liquid emulsion has a pH of approximately 7.4. The liquid emulsion is dried by rotary evaporation to produce a dry powder.

Although a combination of features is shown in the illustrated examples, not all of them need to be combined to realize the benefits of various embodiments of this disclosure. In other words, a system designed according to an embodiment of this disclosure will not necessarily include all of the features shown in any one of the Figures or all of the portions schematically shown in the Figures. Moreover, selected features of one example embodiment may be combined with selected features of other example embodiments.

The preceding description is exemplary rather than limiting in nature. Variations and modifications to the disclosed examples may become apparent to those skilled in the art that do not necessarily depart from this disclosure. The scope of legal protection given to this disclosure can only be determined by studying the following claims.

What is claimed is:

1. A method of preparing a cannabinoid-containing additive, comprising:
preparing an oil mixture by combining a cannabinoid oil and a carrier oil;
preparing a water base by combining water and an emulsifier blend; and
combining the oil mixture and the water base to form a cannabinoid emulsion.

2. The method as recited in claim 1, wherein the cannabinoid oil is cannabidiol (CBD) oil.

3. The method as recited in claim 1, wherein the carrier oil is selected from the group consisting of coconut oil, olive oil, soybean oil, grapeseed oil, avocado oil, and combinations thereof.

4. The method as recited in claim 1, wherein the emulsifier blend is two or more emulsifiers selected from the group consisting of rhamnolipid, xanthan gum, lecithin, guar gum, quillaja saponin, karaya gum, and acacia gum.

5. The method as recited in claim 4, wherein the emulsifier blend is lecithin, karaya gum, and quillaja saponin.

6. The method as recited in claim 4, wherein the emulsifier blend is lecithin, acacia gum, and quillaja saponin.

7. The method as recited in claim 4, wherein the emulsifier blend is rhamnolipid, xanthan gum, and lecithin.

8. The method as recited in claim 4, wherein the emulsifier blend is rhamnolipid, guar gum, and lecithin.

9. The method as recited in claim 4, wherein, by weight, at least half of the emulsifier blend is at least one of rhamnolipid or quillaja saponin.

10. The method as recited in claim 4, wherein, by weight, the emulsifier that is present in the emulsifier blend in is at least one of rhamnolipid or quillaja saponin.

11. The method as recited in claim 1, further including sonicating the water base, and wherein the combining includes adding the oil mixture to the water base during the sonicating.

12. The method as recited in claim 1, further including removing the water from the cannabinoid emulsion to form a dry powder.

13. The method as recited in claim 1, further including either adding the cannabinoid emulsion to a food and beverage product or removing the water from the cannabinoid emulsion to form a dry powder and then adding the dry powder to the food and beverage product.

14. The method as recited in claim 1, further including adding a bioenhancer, the bioenhancer selected from the group consisting of curcumin and piperine.

15. A cannabinoid-containing additive comprising, by weight percent:
1% to 5% of an emulsifier blend;
1% to 15% of a cannabinoid oil;
8% to 12% of a carrier oil; and
a remainder of water.

16. The cannabinoid-containing additive as recited in claim 15, wherein the cannabinoid oil is cannabidiol (CBD) oil.

17. The cannabinoid-containing additive as recited in claim 15, wherein the carrier oil is selected from the group consisting of coconut oil, olive oil, soybean oil, grapeseed oil, avocado oil, and combinations thereof.

18. The cannabinoid-containing additive as recited in claim 15, wherein the emulsifier blend is two or more emulsifiers selected from the group consisting of rhamnolipid, xanthan gum, lecithin, guar gum, quillaja saponin, karaya gum, and acacia gum.

19. The cannabinoid-containing additive as recited in claim 18, wherein the emulsifier blend is lecithin, karaya gum, and quillaja saponin.

20. The cannabinoid-containing additive as recited in claim 18, wherein the emulsifier blend is lecithin, acacia gum, and quillaja saponin.

21. The cannabinoid-containing additive as recited in claim 18, wherein the emulsifier blend is rhamnolipid, xanthan gum, and lecithin.

22. The cannabinoid-containing additive as recited in claim 18, wherein the emulsifier blend is rhamnolipid, guar gum, and lecithin.

23. The cannabinoid-containing additive as recited in claim 15, further including a bioenhancer, the bioenhancer selected from the group consisting of curcumin and piperine.

24. A consumer product comprising:
a food and beverage product including either a cannabinoid-containing additive comprising, by weight percent: 1% to 5% of an emulsifier blend, 1% to 15% of a cannabinoid oil, 8% to 12% of a carrier oil, and a remainder of water, or a dried powder of the cannabinoid-containing additive without the water.
